(19) United States
(12) Patent Application Publication
(10) Pub. No.: US 2026/0144759 A1
(43) Pub. Date: May 28, 2026

(54) Surface-modified plant-derived extracellular vesicles and targeted drug delivery system using the same

(71) Applicant: KOREA INSTITUTE OF SCIENCE AND TECHNOLOGY, Seoul (KR)

(72) Inventors: KYUNGHWAN MOON, Seoul (KR); JI YOON KANG, Seoul (KR)

(73) Assignee: KOREA INSTITUTE OF SCIENCE AND TECHNOLOGY, Seoul (KR)

(21) Appl. No.: 18/725,062

(22) PCT Filed: Dec. 30, 2022
(86) PCT No.: PCT/KR2022/021749

§ 371 Date: Jun. 27, 2024

(30) Foreign Application Priority Data
Dec. 30, 2022 (KR) 10-2022-0190485

(51) Int. Cl. A61K 9/50; A61K 47/54; A61K 47/69

(52) U.S. Cl. CPC A61K 9/5063; A61K 47/544; A61K 47/549; A61K 47/6925

(57) ABSTRACT

The present invention relates to a plant-derived extracellular vesicle surface-modified with a phospholipid including a functional group and a targeting material, a method for producing the same, and a drug delivery vehicle or pharmaceutical composition using the same, and the extracellular vesicle can be stabilized and mass-produced, and thus can be used for intracellular material delivery.

### CROSS-REFERENCE TO RELATED APPLICATIONS

This application is a U.S. National Stage Application of International Application No. PCT/KR2022/021749, filed on Dec. 30, 2022, which claims the benefit of Korean Patent Application No. 10-2022-0190485 filed on Dec. 30, 2022, in the Korean Intellectual Property Office, the entire disclosures of which are incorporated herein by reference for all purposes.

### TECHNICAL FIELD

The present invention relates to a plant-derived extracellular vesicle surface-modified with a phospholipid, a method for producing the same, and a drug delivery vehicle or pharmaceutical composition using the same.

### BACKGROUND ART

Extracellular vesicles refer to membrane particles surrounded by a small lipid bilayer that includes apoptotic bodies, microvesicles, and exosomes. Extracellular vesicles are secreted from humans and animals as well as insects, plants, and microbial cells. In particular, extracellular vesicles include specific molecules possessed by cells, such as proteins, nucleic acids, lipids, and carbohydrates, are surrounded by a lipid bilayer, and thus, are characterized by being able to deliver these molecules to other cells after secretion while stably protecting these proteins. Extracellular vesicles not only transport the various signaling molecules described above, but also act as functional molecules in the microenvironment beyond cell boundaries. Therefore, recently, many studies have been conducted on extracellular vesicles as a drug delivery platform for disease treatment. However, animal-derived extracellular vesicles used in the related art have a stability problem and a disadvantage in that high costs may be required during mass production.

As an alternative, plant-derived extracellular vesicles play an important role in mediating intercellular communication by delivering biomolecules including proteins and RNA to recipient cells through the extracellular space, similar to extracellular vesicles derived from animals such as mammals, and may be transformed into a means for delivery of therapeutic agents such as miRNA and drugs. However, in the case of plant-derived extracellular vesicles, a marker factor for recognizing a target in the human body is not present. Therefore, there is a need for research into techniques for modifying the surface of a plant-derived extracellular vesicle, such that the plant-derived extracellular vesicle can have such marker factors, or for processing the plant-derived extracellular vesicle, such that the plant-derived extracellular vesicle includes a targeting moiety.

### DISCLOSURE

### Technical Problem

Therefore, in order to provide a plant-derived extracellular vesicle capable of replacing the animal cell-derived extracellular vesicle conventionally used as a drug delivery vehicle, the present inventors provided a surface moiety including a functional group such as a maleimide group such that the surface of the plant-derived extracellular vesicle could be modified with a specific material, and completed the present invention for a plant-derived extracellular vesicle whose surface has been modified, such that a drug was targeted in the human body and delivered, or could be recognized at a specific site.

Therefore, an object of the present invention is to provide a plant-derived extracellular vesicle surface-modified with a phospholipid including a functional group.

Another object of the present invention is to provide a drug delivery vehicle including the extracellular vesicle.

Still another object of the present invention is to provide a pharmaceutical composition including the extracellular vesicle.

Yet another object of the present invention is to provide a method for modifying the surface of a plant-derived extracellular vesicle with a phospholipid including a functional group.

### Technical Solution

To achieve the objects described above, the present invention provides a plant-derived extracellular vesicle surface-modified with a phospholipid including a functional group.

To achieve another object of the present invention, the present invention provides a drug delivery vehicle including the extracellular vesicle.

To achieve still another object of the present invention, the present invention provides a pharmaceutical composition including the extracellular vesicle.

To achieve yet another object of the present invention, the present invention provides a method for modifying the surface of a plant-derived extracellular vesicle with a phospholipid including a functional group.

### Advantageous Effects

The present invention relates to a plant-derived extracellular vesicle surface-modified with a phospholipid including a functional group and a targeting material, a method for producing the same, and a drug delivery vehicle or pharmaceutical composition using the same, and has an effect in which an economical and stable extracellular vesicle is efficiently produced, and can be utilized to deliver various drugs.

### DESCRIPTION OF DRAWINGS

FIG. 1 is a schematic view illustrating the process of producing a plant-derived extracellular vesicle whose surface has been modified according to the present invention.

FIG. 2 shows the results of confirming the results of binding a targeting material to the phospholipid functional group of the plant-derived extracellular vesicle whose surface has been modified according to the present invention through the binding of a fluorescent substance.

FIGS. 3A-3C show the results of observing changes in size of the plant-derived extracellular vesicle whose surface has been modified according to the present invention before and after surface modification.

FIG. 4 shows the results of comparing the efficiency of delivery to an intracellular target when the plant-derived extracellular vesicle whose surface has been modified according to the present invention includes a targeting material.

### MODES OF THE INVENTION

To achieve the objects described above, the present invention provides a plant-derived extracellular vesicle surface-modified with a phospholipid including a functional group.

To achieve another object of the present invention, the present invention provides a drug delivery vehicle including the extracellular vesicle.

To achieve still another object of the present invention, the present invention provides a pharmaceutical composition including the extracellular vesicle.

To achieve yet another object of the present invention, the present invention provides a method for modifying the surface of a plant-derived extracellular vesicle with a phospholipid including a functional group.

Hereinafter, the present invention will be described in detail.

As an aspect of the present invention, the present invention relates to a plant-derived extracellular vesicle surface-modified with a phospholipid including a functional group.

As used herein, the term “extracellular vesicle” refers to a small vesicle with a membrane structure secreted by various cells, and is characterized by having a diameter in a range of approximately 30 to 1,000 nm and being released into the extracellular environment through the fusion of a multivesicular body and a plasma membrane. Extracellular vesicles may be cell membrane-derived vesicles, ectosomes, shedding vesicles, microparticles, exosomes, microvesicles, outer membrane vesicles, and apoptotic bodies, and in particular, the extracellular vesicles in the present invention may be exosomes.

The plant-derived vesicle of the present invention may be particularly derived from edible plants, and not only contains useful plant components, but also has few side effects as a natural material and can be delivered and applied orally into the intestines.

In the present specification, the extracellular vesicle is one whose surface has been modified with a phospholipid including a functional group. The type of phospholipid is not limited, and the phospholipid may be a phospholipid selected from the group consisting of, for example, dipalmitoylphosphatidylcholine (DPPC), distearoylphosphatidylcholine (DSPC), phosphocholine (DOPC), dimyristoylphosphatidylcholine (DMPC), phosphatidylcholine (PLPC), phosphatidylethanolamine (PE), egg phosphatidylcholine (EPC), dilauroyl phosphatidylcholine (DLPC), dimyristoylphosphatidylcholine (DMPC), 1-myristoyl-2-palmitoyl phosphatidylcholine (MPPC), 1-palmitoyl-2-myristoyl phosphatidylcholine (PMPC), 1-palmitoyl-2-stearoylphosphatidylcholine (PSPC), 1-stearoyl-2-palmitoyl phosphatidylcholine (SPPC), 1,2-distearoyl-sn-glycero-3-phosphocholine (DAPC), 1,2-dialachidoyl-sn-glycero-3-phosphocholine (DBPC), 1,2-dieicosenoyl-sn-glycero-3-phosphocholine (DEPC), palmitoyl oleoyl phosphatidylcholine (POPC), lysophosphatidylcholine, dilinoleoylphosphatidylcholine, distearoylphosphatidylethanolamine (DSPE), dimyristoyl phosphatidylethanolamine (DMPE), dipalmitoyl phosphatidylethanolamine (DPPE), palmitoyl oleoyl phosphatidylethanolamine (POPE), and lysophosphatidylethanolamine.

Further, the phospholipid may be a PEG lipid including a PEG derivative. A “lipid-polyethylene glycol (PEG) conjugate”, “lipid-PEG”, “PEG-lipid”, or “lipid-PEG” refers to a form in which a lipid and PEG are conjugated, and means a lipid with a polyethylene glycol (PEG) polymer, which is a hydrophilic polymer, bound to one end thereof. In addition, the PEG may be a PEG with a functional group bound to the side that is not bound to lipids (functionalized PEG). In this case, an available functional group may be one or more selected from the group consisting of a succinyl group, a carboxylic acid, maleimide, an amine group, biotin, a cyanur group, folate, and the like.

In an exemplary embodiment of the present invention, DSPE-PEG2000-maleimide (1,2-distearoyl-sn-glycero-3-phosphoethanolamine-N-[maleimide (polyethylene glycol)-2000] in which a phospholipid DSPE and a PEG2000-maleimide group as a functional group are bound was used to modify the surface of the extracellular vesicle. The DSPE-PEG2000-maleimide may be represented by the following chemical formula.

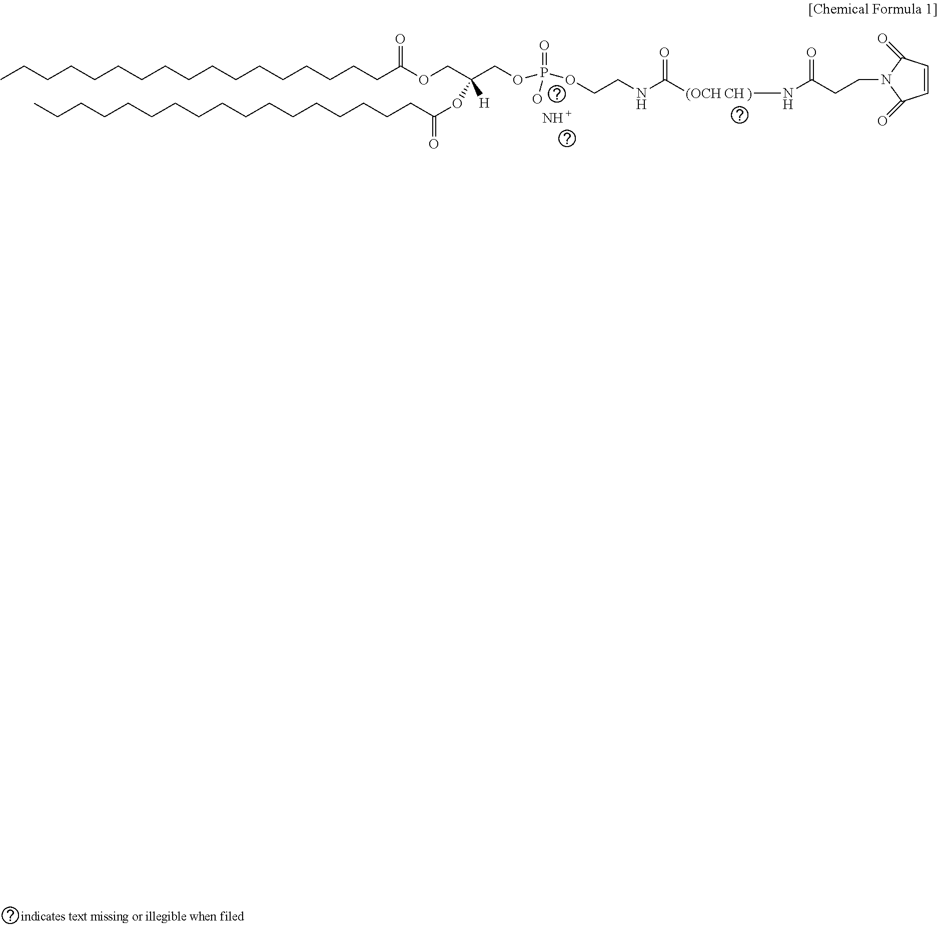

In addition, the extracellular vesicle of the present invention may further include a targeting material for targeting. The targeting material is not limited to a type such as a peptide, a nucleic acid, an aptamer, and a chemical ligand, and may be bound to the surface through a click chemistry reaction. In an exemplary embodiment of the present invention, an aptamer was used as a targeting material. The “aptamer” refers to an oligonucleotide (generally 20 to 80 nt DNA or RNA) that binds to a specific target.

The aptamer may modify the membrane structure by being bound to a functional group bound to the phospholipid on the surface of the extracellular vesicle of the present invention, and may be bound through a click chemistry reaction for a stable and efficient reaction. For example, as in an exemplary embodiment of the present invention, the aptamer includes a thiol group and may be bound through a maleimide group on the surface of the extracellular vesicle.

As another aspect of the present invention, the present invention provides a drug delivery vehicle or a pharmaceutical composition including the plant-derived extracellular vesicle whose surface has been modified.

As used herein, the “drug delivery vehicle” refers to a pharmaceutical composition containing one or more therapeutic drug materials or nucleic acid molecules, which are administered to a mammal, such as a human. A pharmaceutical composition is one that is “pharmaceutically acceptable,” meaning a compound, material, composition and/or dosage form suitable for contact with mammalian, especially human tissue without undue toxicity, irritation, allergic reactions, and other complications commensurate with the reasonable benefit/risk within the scope of sound medical judgment.

In the present invention, the extracellular vesicle may be used as a drug delivery vehicle capable of carrying and delivering a polypeptide, a peptide, a protein, a membrane receptor, mRNA, siRNA, miRNA, DNA, a compound, and the like.

Furthermore, the present invention relates to a pharmaceutical composition including the plant-derived extracellular vesicle whose surface has been modified, and the pharmaceutical composition refers to a material used for the prevention or treatment of a disease.

As used herein, the term “prevention” refers to all actions capable of suppressing a disease or delaying the onset of the disease by administering the pharmaceutical composition according to the present invention.

As used herein, the term “treatment” refers to all actions that ameliorate or beneficially change symptoms of a disease by administering the pharmaceutical composition according to the present invention.

The pharmaceutical composition of the present invention may be used by being formulated in the form of an oral formulation, such as a powder, a granule, a pill, a capsule, a suspension, an emulsion, a syrup, and an aerosol, an external preparation, a suppository, and a sterile injection solution, according to a typical method, and may further include a carrier, an excipient, or the like required for formulation. Examples of pharmaceutically acceptable carriers, excipients or diluents, which may be further included in the pharmaceutical composition, include lactose, dextrose, sucrose, sorbitol, mannitol, xylitol, erythritol, maltitol, starch, acacia rubber, alginate, gelatin, calcium phosphate, calcium silicate, cellulose, methyl cellulose, microcrystalline cellulose, polyvinyl pyrrolidone, water, methyl hydroxybenzoate, propyl hydroxybenzoate, talc, magnesium stearate, mineral oil, and the like. When the pharmaceutical composition is prepared, the pharmaceutical composition is prepared using a diluent or excipient, such as a filler, an extender, a binder, a wetting agent, a disintegrant, and a surfactant, which are commonly used.

For example, a solid preparation for oral administration includes a tablet, a pill, a powder, a granule, a capsule, and the like, and the solid formulation is prepared by mixing at least one excipient, for example, starch, calcium carbonate, sucrose or lactose, gelatin, and the like with the extract or compound. Further, in addition to a simple excipient, lubricants such as magnesium stearate and talc are also used. A liquid formulation for oral administration corresponds to a suspension, a liquid for internal use, an emulsion, a syrup, or the like, and the liquid formulation may include, in addition to water and liquid paraffin, which are simple commonly used diluents, various excipients, for example, a wetting agent, a sweetener, a fragrance, a preservative, and the like.

Examples of a formulation for parenteral administration include an aqueous sterile solution, a non-aqueous solvent, a suspension, an emulsion, a freeze-dried preparation, and a suppository. As the non-aqueous solvent and the suspension, it is possible to use propylene glycol, polyethylene glycol, a vegetable oil such as olive oil, an injectable ester such as ethyl oleate, and the like. As a base of the suppository, it is possible to use Witepsol, Macrogol, Tween 61, cacao butter, laurin fat, glycerogelatin, and the like.

The pharmaceutical composition of the present invention may be orally administered or may be parenterally administered (intravenously, subcutaneously, intraperitoneally, or topically applied), and the administration dose may vary depending on a patient's condition and body weight, the severity of disease, drug form, and administration route and period according to the desired method, and the administration dose may be selected in an appropriate form by those skilled in the art.

The pharmaceutical composition of the present invention is administered in a pharmaceutically effective amount. In the present invention, the “pharmaceutically effective amount” refers to an amount sufficient to treat diseases as an amount applicable to medical treatment, and the criteria thereof may be determined according to types of diseases of patients, the severity of disease, the activity of drugs, sensitivity to drugs, administration time, administration route, excretion rate, treatment period, ingredients used in combination, and other items. The pharmaceutical composition of the present invention may be administered as an individual therapeutic agent or may be administered in combination with other therapeutic agents, and may be administered sequentially or simultaneously with therapeutic agents in the related art. The administration dose may be determined at a level that can minimize side effects in consideration of all of the above factors, and may be easily determined by those skilled in the art. Specifically, the administration dose of the pharmaceutical composition may vary depending on the patient's age, body weight, severity, sex, and the like, and generally, 0.001 to 150 mg of the pharmaceutical composition and more preferably, 0.01 to 100 mg of the pharmaceutical composition, per 1 kg of the body weight, may be administered daily or every other day, or one to three times a day. However, this is exemplary, and the administration dose may be set differently, if necessary.

As still another aspect of the present invention, the present invention relates to a method for modifying the surface of a plant-derived extracellular vesicle with a phospholipid including a functional group, and includes the following steps.
1) obtaining an extracellular vesicle from a plant;
2) modifying the surface of the obtained extracellular vesicle with a phospholipid including a functional group; and
3) binding a targeting material through a click chemistry reaction.

Step 1) above may be performed by a known method for obtaining a vesicle through a pulverized material, extract, and the like of a plant, and may be performed by centrifuging, for example, a pulverized plant material, juice, or extract to obtain a residual pellet. The process is not limited to a method, and ultracentrifugation, size exclusion chromatography or an extracellular vesicle isolation kit may be used.

In Step 2) above, the modifying of the surface of the extracellular vesicle may use various types of phospholipids mentioned above, and preferably, may modify the surface using DPSE-PEG (2000)-maleimide.

The type of targeting material in Step 3) above may vary depending on the target of a drug to be carried, and the targeting material may be included without limitation as long as it can bind to the surface-modified functional group of the extracellular vesicle through a click chemistry reaction.

For an extracellular vesicle whose surface has been modified with a phospholipid, a functional group is exposed to the outside of the vesicle. The phospholipid of the DPSE-PEG (2000)-maleimide of an exemplary embodiment of the present invention is located on the surface membrane of the extracellular vesicle, and in this case, DSPE is located between the phospholipids that constitute the surface of the extracellular vesicle, and the PEG2000-maleimide moiety is exposed to the outside of the extracellular vesicle. When a targeting material capable of imparting specific targeting ability includes a —SH group (thiol group), if the targeting material is incubated with a surface-modified extracellular vesicle in which the PEG2000-maleimide group is exposed to the outside, the maleimide group and the thiol group bind through click chemistry, resulting in the binding of the targeting material to the surface of the extracellular vesicle.

For the binding of the surface modifying material and the targeting material in the extracellular vesicle, the type of functional group is not limited as long as the functional group is bound through click chemistry.

Hereinafter, the present specification will be described in detail with reference to Examples in order to specifically explain the present specification. However, the Examples according to the present specification may be modified into various forms, and it should not be interpreted that the scope of the present specification is limited to the Examples described below in detail. The Examples of the present specification are provided to more completely describe the present specification to a person with ordinary skill in the art.

### Example 1. Isolation and Surface Modification of Plant-Derived Extracellular Vesicles

1-1. Isolation of Extracellular Vesicles from Plant

Extracellular vesicles were isolated from juiceable grapefruit. After the grapefruit was washed, fruit juice was squeezed from the grapefruit using a juice squeezer, then 500 g, 3,000 g, and 10,000 g were sequentially applied to the fruit juice using a centrifuge, debris were removed, and large materials other than extracellular vesicles present in the fruit juice were removed using a 0.2 μm filter. The fruit juice from which large materials had been removed was subjected to ultrahigh-speed centrifugation at 100,000 g or more, and extracellular vesicles were isolated using size exclusion chromatography and an extracellular vesicle isolation kit

### 1-2. Surface Modification of Isolated Extracellular Vesicles

The isolated extracellular vesicles were dispersed in PBS at a desired concentration. Thereafter, after the dispersed extracellular vesicles were mixed with DSPE-PEG2000-maleimide dissolved in EtOH and incubated at room temperature or 4° C., excess materials that did not participate in the modification were removed using a 100 kD filter, a dialysis tube, and ultrahigh-speed centrifugation, and only the surface-modified extracellular vesicles were isolated.

### 1-3. Binding of Targeting Material

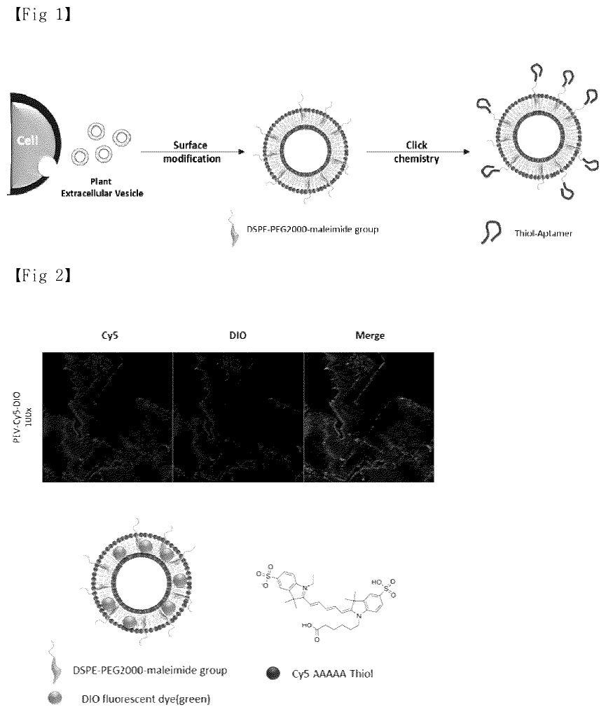

For extracellular vesicles whose surface has been modified with a phospholipid (DSPE-PEG2000-maleimide), a functional group is exposed to the outside of the vesicle. The phospholipid of the DPSE-PEG2000-maleimide group of the present example is located on the surface membrane of the extracellular vesicle, and in this case, DSPE is located between the phospholipids that constitute the surface of the extracellular vesicle, and the PEG2000-maleimide moiety is exposed to the outside of the extracellular vesicle. An aptamer was incubated with the extracellular vesicles, such that the externally exposed maleimide group and the —SH group of the aptamer were bound. In this case, the maleimide group and thiol group were bound through click chemistry, imparting targeting ability to the extracellular vesicles. The process is shown in FIG. 1.

### Example 2. Confirmation of Surface Modification of Plant-Derived Extracellular Vesicles

### 2-1. Fluorescent Imaging Analysis

Fluorescence imaging analysis was performed to confirm whether the surface of the surface-modified extracellular vesicles from Example 1 above had been modified. A green fluorescent material DiO is a hydrophobic material and located between the phospholipid bilayer of the extracellular vesicle, and a red fluorescent material Cy5 was bound to a thiol group and incubated with a grapefruit extracellular vesicle whose surface had been modified. As a result, the maleimide group and the thiol group were bound through click chemistry, and after unbound materials were removed, it was confirmed whether the maleimide group and the thiol group were bound using a fluorescence microscope.

As a result, as shown in FIG. 2, it was confirmed that the red Cy5 material and the green DiO material were observed at the same position, and completely overlapped. Through the results described above, it could be confirmed that the surface of the extracellular vesicle was modified.

### 2-2. Measurement of Size of Surface-Modified Extracellular Vesicles

The size of the extracellular vesicles before and after surface modification was confirmed using a dynamic light scattering (DLS) apparatus. The sizes of a plant-derived extracellular vesicle (pEV), and pEV-R113 in which an aptamer was attached through click chemistry after the plant-derived extracellular vesicle was substituted with DSPE-PEG (2000)-maleimide and pEV-random were compared.

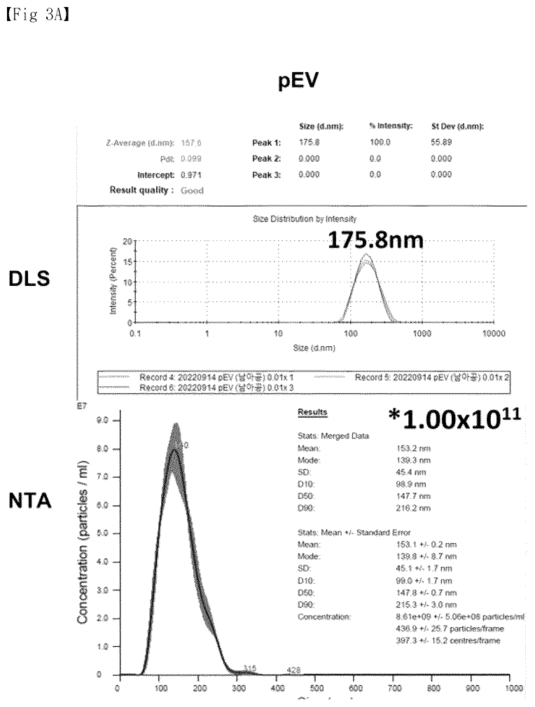

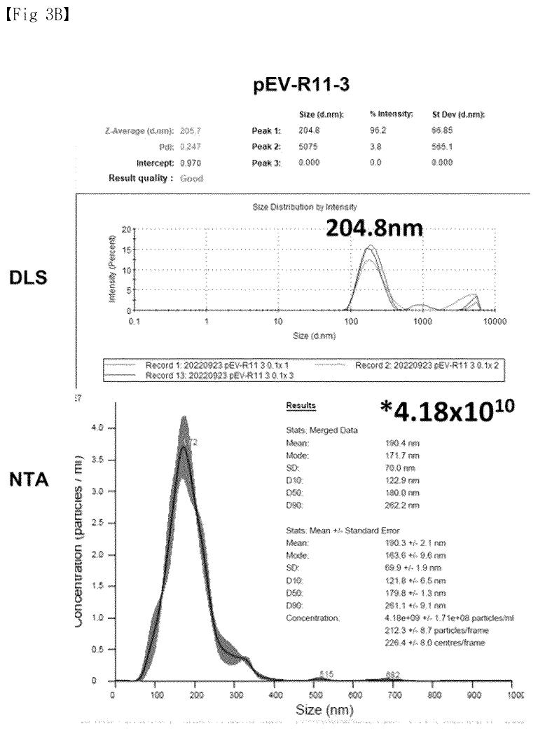

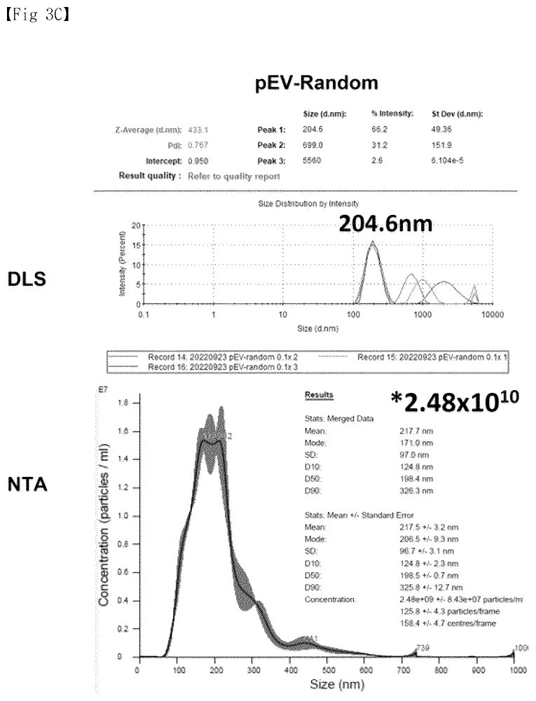

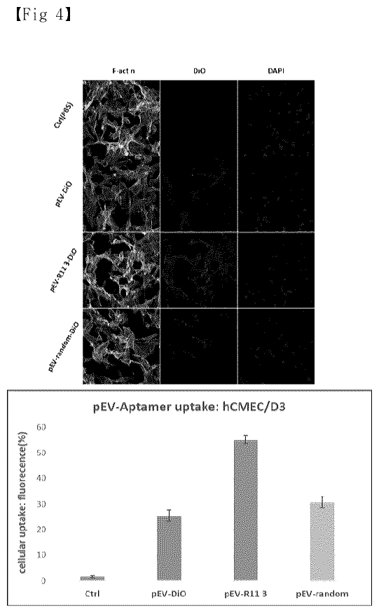

As a result, as shown in FIGS. 3A-3C, before surface modification, the extracellular vesicles exhibited an average size of around 175 nm, and when an aptamer was attached through click chemistry after modification, the extracellular vesicles exhibited a size of around 204 nm. From this, it can be confirmed through the size that the surface of the extracellular vesicle is substituted with the aptamer.

### Example 3. Confirmation of Intracellular Delivery of Plant-Derived Extracellular Vesicles

In order to confirm the intracellular delivery ability of the plant-derived extracellular vesicles whose surface had been modified, DiO, which is a green fluorescent material, was added to the plant-derived extracellular vesicles and the plant-derived extracellular vesicles whose surface had been modified, respectively, and then the vesicles were absorbed into hCMEC/D3 cells, which are endothelial cells, and the difference was confirmed using a fluorescence microscope.

The degree to which the plant-derived extracellular vesicles had entered cells could be confirmed through DiO, which is a green fluorescent material, and it could be confirmed that when an aptamer, which is a targeting material, was attached to the extracellular vesicles whose surface had been modified through click chemistry (pEV-R113), a larger amount of vesicles was introduced into the cells compared to the case where the aptamer was not attached (pEV-DiO) (FIG. 4).

In the foregoing, the present invention has been examined mainly based on the preferred examples thereof. A person with ordinary skill in the art to which the present invention pertains will be able to understand that the present invention may be implemented in a modified form without departing from the essential characteristics of the present invention. Therefore, the disclosed examples should be considered not from a restrictive viewpoint, but from an explanatory viewpoint. The scope of the present invention is defined not in the above-described description, but in the claims, and it should be interpreted that all the differences within a range equivalent thereto are included in the present invention.

1. A plant-derived extracellular vesicle whose surface has been modified with a phospholipid comprising a functional group to which a targeting material can bind.

2. The extracellular vesicle of claim 1, wherein the phospholipid is selected from the group consisting of dipalmitoylphosphatidylcholine (DPPC), phosphocholine (DOPC), dimyristoylphosphatidylcholine (DMPC), phosphatidylcholine (PLPC), distearoylphosphatidylcholine (DSPC), phosphatidylethanolamine (PE), egg phosphatidylcholine (EPC), dilauroyl phosphatidylcholine (DLPC), dimyristoylphosphatidylcholine (DMPC), 1-myristoyl-2-palmitoyl phosphatidylcholine (MPPC), 1-palmitoyl-2-myristoyl phosphatidylcholine (PMPC), 1-palmitoyl-2-stearoylphosphatidylcholine (PSPC), 1-stearoyl-2-palmitoyl phosphatidylcholine (SPPC), 1,2-distearoyl-sn-glycero-3-phosphocholine (DAPC), 1,2-dialachidoyl-sn-glycero-3-phosphocholine (DBPC), 1,2-dieicosenoyl-sn-glycero-3-phosphocholine (DEPC), palmitoyl oleoyl phosphatidylcholine (POPC), lysophosphatidylcholine, dilinoleoylphosphatidylcholine, distearoylphosphatidylethanolamine (DSPE), dimyristoyl phosphatidylethanolamine (DMPE), dipalmitoyl phosphatidylethanolamine (DPPE), palmitoyl oleoyl phosphatidylethanolamine (POPE), and lysophosphatidylethanolamine.

3. The extracellular vesicle of claim 1, wherein the phospholipid is bound to a PEG derivative comprising a functional group.

4. The extracellular vesicle of claim 1, wherein the phospholipid is DSPE-PEG2000-maleimide.

5. The extracellular vesicle of claim 1, further comprising a targeting material.

6. The extracellular vesicle of claim 5, wherein the targeting material is any one of a peptide, a nucleic acid, an aptamer, or a chemical ligand.

7. The extracellular vesicle of claim 1, wherein the extracellular vesicle is an exosome.

8. A drug delivery vehicle comprising the extracellular vesicle of claim 1.

9. A pharmaceutical composition comprising the extracellular vesicle of claim 1.

10. A method for modifying an extracellular vesicle, the method comprising:
1) obtaining an extracellular vesicle from a plant;
2) modifying the surface of the obtained extracellular vesicle with a phospholipid including a functional group; and
3) binding a targeting material through a click chemistry reaction.

11. The method of claim 10, wherein the phospholipid in Step 2) is DSPE-PEG2000-maleimide.

12. The method of claim 10, wherein the targeting material in Step 3) is an aptamer comprising a thiol group.
